SEQUENCE LISTING (16 sequences)

SEQ ID NO: 1 - DNA, 29 bp, Artificial Sequence
  Description of Artificial Sequence Common probe
ggccagtaagtggcagacttggtgaggtt

SEQ ID NO: 2 - DNA, 29 bp, Artificial Sequence
  Description of Artificial Sequence Common2 probe
ggccagtaactggcagacttggtgaggtt

SEQ ID NO: 3 - DNA, 15 bp, Artificial Sequence
  Description of Artificial Sequence Query probe
gccacttactggccc

SEQ ID NO: 4 - DNA, 15 bp, Artificial Sequence
  Description of Artificial Sequence Query2 probe
gccacttactgcccc

SEQ ID NO: 5 - DNA, 41 bp, Artificial Sequence
  Description of Artificial Sequence 5′-sense (C) probe
ccgtgtccactctagaaaaacctgcattgccagtgtagtcg

SEQ ID NO: 6 - DNA, 41 bp, Artificial Sequence
  Description of Artificial Sequence 5′-sense (T) probe
accaccgcttgaatacaaaacatgcattgccagtgtagtca

SEQ ID NO: 7 - DNA, 52 bp, Artificial Sequence
  Description of Artificial Sequence 3′-sense probe
ggccagtaactggcagacttggtgaggttcatctaaagcgttcccagttcca

SEQ ID NO: 8 - DNA, 38 bp, Artificial Sequence
  Description of Artificial Sequence 5′-antisense (C) probe
ccgtgtccactctagaaaaacctgccacttactgcccc

SEQ ID NO: 9 - DNA, 40 bp, Artificial Sequence
  Description of Artificial Sequence 5′-antisense (T) probe
accaccgcttgaatacaaaacatctgccacttactgccct

SEQ ID NO: 10 - DNA, 54 bp, Artificial Sequence
  Description of Artificial Sequence 3′-antisense probe
gagtacactgccaatccagaagcagatgcccgcagattcattggtcagagaaca

SEQ ID NO: 11 - DNA, 23 bp, Artificial Sequence
  Description of Artificial Sequence Tag sequence of rED
ccgtgtccactctagaaaaacct

SEQ ID NO: 12 - DNA, 23 bp, Artificial Sequence
  Description of Artificial Sequence Tag sequence of rED′
accaccgcttgaatacaaaacat

SEQ ID NO: 13 - DNA, 23 bp, Artificial Sequence
  Description of Artificial Sequence Tag sequence of rDCN1
gcagattcattggtcagagaaca

SEQ ID NO: 14 - DNA, 23 bp, Artificial Sequence
  Description of Artificial Sequence Tag sequence of rDCN2
catctaaagcgttcccagttcca

SEQ ID NO: 15 - DNA, 40 bp, Artificial Sequence
  Description of Artificial Sequence Forward primer
ttacatgcaagggtttccataaggtctacacctgagggtc

SEQ ID NO: 16 - DNA, 41 bp, Artificial Sequence
  Description of Artificial Sequence Reverse primer
atgtcacatgcaaagagattccaccagttccattatcctct

(19) United States
(12) Patent Application Publication
(10) Pub. No.: US 2011/0117547 A1
(43) Pub. Date: May 19, 2011

(54) TARGET DNA DETECTION METHOD AND TARGET DNA DETECTION KIT

(75) Inventor: Tetsuya Tanabe, Tokyo (JP)

(73) Assignee: OLYMPUS CORPORATION, Tokyo (JP)

(21) Appl. No.: 12/674,495

(22) PCT Filed: Oct. 21, 2008
(86) PCT No.: PCT/JP2008/069015

§ 371 Date: Feb. 22, 2010

(30) Foreign Application Priority Data
Oct. 23, 2007 (JP) 2007-275698

(51) Int. Cl. C12Q 1/68

(52) U.S. Cl. 435/6

(57) ABSTRACT

The present invention provides a method for detecting a target DNA easily and highly accurately through simultaneous analysis of a sense strand and an antisense strand of the target DNA, and a kit therefor. The target DNA detection method of the present invention is a method for detecting a target DNA composed of a sense strand having a target nucleotide sequence and an antisense strand complementary to the sense strand, wherein: a first oligonucleotide which hybridizes with the sense strand, and a second oligonucleotide which hybridizes with the antisense strand are used; at least a part of a region, of the antisense strand, which hybridizes with the second oligonucleotide is complementary to at least a part of a region, of the sense strand, which hybridizes with the first oligonucleotide; and the first oligonucleotide and the second oligonucleotide are simultaneously added into one reaction solution to effect respective hybridizations with the target DNA, followed by ligation reactions and/or PCR, thereby detecting resultant products.

### TECHNICAL FIELD

The present invention relates to a method to which the hybridization method is applied for accurately detecting a target DNA through simultaneous analysis of a sense strand having a target nucleotide sequence and an antisense strand complementary to this sense strand, and a kit for use in this method.

### BACKGROUND ART

With the recent progress in genetic engineering technologies and the gene recombination technologies, genetic tests through nucleic acid analyses have been widely used for applications to medical services, researches, and industries. Such tests are to detect the presence of DNA which has a target nucleotide sequence in a sample, and have been applied not only to diagnosis and treatment of diseases, but also in various fields such as food inspection. In particular, SNP (Single Nucleotide Polymorphism) is considered to be a major factor for individual differences in the vulnerability against a specific disease such as cancer, the drug metabolizing capacity, and so forth. SNP analyses have been widely performed not only in researches but also in actual clinical tests. Therefore, highly accurate and quick methods for detecting SNP have been enthusiastically developed.

As methods for detecting SNP, many methods have been reported in which artificially synthesized short chain oligonucleotides such as probes and primers are used to examine the nucleotide sequences of nucleic acids. For example, usually employed methods are such that SNP serving as the detection target and its neighboring nucleotide sequences are analyzed through molecular-biological enzymatic reactions, likewise of methods for detecting SNP by the presence/absence of a PCR product through PCR (Polymerase Chain Reaction) using a primer that is specifically bindable to a certain allele in a given SNP, and methods for detecting SNP by the obtainability of an oligonucleotide bound with two probes through a ligation reaction using a probe having the detection target SNP at the 3′ end and a probe having a nucleotide adjacent to the 5′ side of the SNP, at the 5′ end.

Such analysis methods utilizing enzymatic reactions involve a problem in that nonspecific reactions easily occur if only one base difference in a nucleotide sequence is to be analyzed likewise of the SNP analysis. For example, in cases where only a limited amount of the analyte sample is available and cases where the sample is problematic in terms of the quality because of lots of impurities or the like, likewise of clinical tests, nonspecific reactions often occur; as a result of which, errors do happen in the analysis. On the other hand, in gene analyses for the medical purpose, test errors may often lead to critical results to the patient, and thus high accuracy has been strongly demanded. Furthermore, in clinical tests, a huge number of specimens often have to be handled in a limited time, and thus the test rapidity and workability have been desired to be improved.

In conventional biochemical tests, error data are detected from biochemical findings through comparison between results of a plurality of items. In SNP detection, similarly to other tests, it is considered that comparative examination between a plurality of test results will bring more accurate results. For example, the nucleotide sequence detection of not only the sense strand but also both strands including the antisense strand can improve the test accuracy because of the complementarity of these bases.

Besides, various methods have been disclosed in order to improve the accuracy of SNP detection. For example, there has been disclosed a method (1) for improving the detection accuracy for a target allele using a modified oligonucleotide probe which is partially complementary to the target nucleic acid sequence, but not completely complementary thereto, so as to increase its affinity to the SNP target allele higher than the affinity to the control allele (for example, refer to Patent Document 1). In addition, there has been a method (2) using a probe which is specifically bindable to a target DNA having a specific nucleotide sequence and is designed to be more stable in a hybrid form with the target DNA than a hybrid form between probes. By lowering the affinity between probes in this manner, nonspecific enzymatic reactions and so forth can be suppressed to improve the accuracy of SNP detection. Such probes are referred to as pseudo-complementary probes. For example, there have been proposed a method using a combination of 2-thiouracil/2,6-diaminopurine (for example, refer to Non-patent Document 1), and an artificial nucleic acid in which the main chain has been altered into PNA (peptide nucleic acid), in addition to introduction of artificial nucleic acids (for example, refer to Non-patent Document 2).

[Patent Document 1] Published Japanese translation No. 2000-511434 of PCT International Publication

[Non-patent Document 1] Kutyavin et al., Biochemistry, Vol. 35, No. 34, pp. 11170-11176 (1996)

[Non-patent Document 2] Lohse et al., Proceedings of the National Academy of Sciences of the United States of America, Vol. 96, No. 21, pp. 11804-11808 (1999)

[Non-patent Document 3] Nishida at al., Analytical Biochemistry, Vol. 364, pp. 78-85 (2007)

### DISCLOSURE OF INVENTION

A probe for analyzing a sense strand and a probe for analyzing an antisense strand corresponding to the analysis region of the sense strand are inevitably complementary to each other. Accordingly, when probes for respectively detecting the sense strand and the antisense strand are simultaneously added to the test target so as to analyze both strands, these probes hybridize with each other, which inhibits hybridizations between the probe and the sense strand, and between the probe and the antisense strand. For this reason, it has been considered that many of such analytical reactions end up in failure. Therefore, in order to prevent the competition of probes, analysis of the sense strand and analysis of the antisense strand need to be each independently performed, which thus brings about problems in the rapidity and the easy workability.

On the other hand, according to the method (2) mentioned above, the stability of the hybrid between a probe and a target DNA can be increased to be higher than the stability of the product between probes; however, use of 2-thiouracil, 2,6-diaminopurine, or other modified nucleic acids, and peptide nucleic acids increments the cost for synthesizing probes, which is not preferable for clinical tests or the like where a large number of specimens need to be handled. Moreover, the purpose of the method (1) mentioned above is to improve the recognition specificity for a nucleotide sequence, and Patent Document 1 does not describe at all regarding the method for simultaneously analyzing both the sense strand and the antisense strand.

It is an object of the present invention to provide a method to which the hybridization method is applied for detecting a target DNA easily and highly accurately through simultaneous analysis of a sense strand and an antisense strand of the target DNA, and a kit for use in the method.

In view of the above problems, the inventors of the present invention have conducted intensive studies. As a result, they have found that a sense strand and an antisense strand can be simultaneously analyzed by performing hybridization through simultaneous addition of an oligonucleotide which hybridizes with the sense strand and an oligonucleotide which hybridizes with the antisense strand into one reaction solution, and that the sense strand and the antisense strand can be simultaneously analyzed with high accuracy by increasing the thermodynamic stabilities of hybrids between these oligonucleotides and the target DNA more than the thermodynamic stability of a hybrid between these oligonucleotides through introduction of appropriate mismatches respectively into these oligonucleotides. This has led to the completion of the present invention.

That is, the present invention provides a method for detecting a target DNA composed of a sense strand having a target nucleotide sequence and an antisense strand complementary to the sense strand, wherein: a first oligonucleotide which hybridizes with the sense strand, and a second oligonucleotide which hybridizes with the antisense strand are used; at least a part of a region, of the antisense strand, which hybridizes with the second oligonucleotide is complementary to at least a part of a region, of the sense strand, which hybridizes with the first oligonucleotide; and the first oligonucleotide and the second oligonucleotide are simultaneously added into one reaction solution to effect respective hybridizations with the target DNA, followed by ligation reactions and/or PCR (Polymerase Chain Reaction), thereby detecting resultant products.

The present invention also provides a target DNA detection method, wherein the number of mismatches in the hybrid between the first oligonucleotide and the second oligonucleotide is greater than the number of mismatches in the hybrid between the sense strand and the first oligonucleotide, or the number of mismatches in the hybrid between the antisense strand and the second oligonucleotide.

The present invention also provides a target DNA detection method, wherein one or more mismatches are respectively introduced into the first oligonucleotide and the second oligonucleotide.

The present invention also provides a target DNA detection method, wherein the target nucleotide sequence is a nucleotide sequence containing a SNP (Single Nucleotide Polymorphism) site.

The present invention also provides a target DNA detection method, wherein: a third oligonucleotide which hybridizes with the sense strand in a region different from the region of the sense strand which hybridizes with the first oligonucleotide, and a fourth oligonucleotide which hybridizes with the antisense strand in a region different from the region of the antisense strand which hybridizes with the second oligonucleotide are used; at least a part of a region, of the antisense strand, which hybridizes with the fourth oligonucleotide is complementary to at least a part of a region, of the sense strand, which hybridizes with the third oligonucleotide; and the first oligonucleotide, the second oligonucleotide, the third oligonucleotide, and the fourth oligonucleotide are simultaneously added into one reaction solution to effect respective hybridizations with the target DNA, followed by ligation reactions and/or PCR (Polymerase Chain Reaction), thereby detecting resultant products.

The present invention also provides a target DNA detection kit for use in any of the target DNA detection methods described above, comprising the first oligonucleotide and the second oligonucleotide.

The present invention also provides a target DNA detection kit for use in the above target DNA detection method, comprising the first oligonucleotide, the second oligonucleotide, the third oligonucleotide, and the fourth oligonucleotide.

Since the target DNA detection method of the present invention is capable of simultaneous analysis of a sense strand and an antisense strand, even in cases where both the sense strand and the antisense strand are analyzed so as to improve the accuracy in SNP detection, a large number of specimens can be easily and rapidly analyzed. In particular, through introduction of appropriate mismatches into oligonucleotides for use in the analysis, the thermodynamic stability of the hybrid between oligonucleotides can be lowered. Therefore, hybridizations between the sense strand and the oligonucleotide, and between the antisense strand and the oligonucleotide can be highly efficiently performed; as a result of which, the SNP detection accuracy can be improved.

### BRIEF DESCRIPTION OF THE DRAWINGS

FIG. 1 shows a concept of one embodiment of the target DNA detection method of the present invention using the DigiTag2 assay.

FIG. 2 shows the nucleotide sequence in the vicinity of rs17822931, wherein [C/T] denotes the SNP site.

FIG. 3 shows the Tm values of respective hybrids measured in Reference Example 1, wherein the left column shows the Tm value of the sense strand-common probe hybrid and the right column shows the Tm value of the common probe-query probe hybrid. In addition, (A) shows a case where the common probe and the query probe were used, (B) shows a case where the common probe and the query 2 probe were used, and (C) shows a case where the common 2 probe and the query 2 probe were used.

FIG. 4 shows the measurement results of the ligation amounts in Example 1, wherein the “Sample type” means the type of SNP of the DNA sample used, and the “NC” means the control. In addition, the “Probe type” means the SNP serving as the analysis target for a probe used, the “C-Probe” means that either one of the 5′ side sense (C) probe or the 5′ side antisense (C) probe was used, and the “T-Probe” means that either one of the 5′ side sense (T) probe or the 5′ side antisense (T) probe was used.

### BRIEF DESCRIPTION OF THE REFERENCE SYMBOLS

1: 5′ side (sense strand) probe, 2: 3′ side (antisense strand) probe, 3: 3′ side (sense strand) probe, 4: 5′ side (antisense strand) probe, 5: oligonucleotide bound with the 5′ side (sense strand) probe 1 and the 3′ side (sense strand) probe 3, 6: oligonucleotide bound with the 3′ side (antisense strand) probe 2 and the 5′ side (antisense strand) probe 4

### BEST MODE FOR CARRYING OUT THE INVENTION

The target nucleotide sequence in the present invention is not specifically limited as long as it can serve as the analysis target and its nucleotide sequence has been identified to an extent that is detectable by gene recombination techniques or the like. Examples thereof may include nucleotide sequences existing in animal or plant chromosomes and bacterial or viral genes, and nucleotide sequences existing in RNAs of a organism such as mRNA. It is preferably a nucleotide sequence containing a SNP site, and more preferably a human nucleotide sequence containing a SNP site.

The target DNA having a target nucleotide sequence in the present invention is a double-stranded DNA composed of a sense strand having the target nucleotide sequence and an antisense strand complementary to the sense strand. Examples of the target DNA may include DNAs contained in biological samples such as blood and body fluids (specimens), DNAs extracted from these biological samples, and DNAs amplified using the above DNAs as templates. In addition, cDNAs synthesized from RNAs contained in biological samples with reverse transcriptases may also be used.

The target DNA detection method of the present invention is a method for detecting a target DNA composed of a sense strand having a target nucleotide sequence and an antisense strand complementary to the sense strand, wherein: a first oligonucleotide which hybridizes with the sense strand, and a second oligonucleotide which hybridizes with the antisense strand are used; at least a part of a region, of the antisense strand, which hybridizes with the second oligonucleotide is complementary to at least a part of a region, of the sense strand, which hybridizes with the first oligonucleotide; and the first oligonucleotide and the second oligonucleotide are simultaneously added into one reaction solution to effect respective hybridizations with the target DNA, followed by ligation reactions and/or PCR, thereby detecting resultant products.

That is, the target DNA detection method of the present invention is characterized in respective hybridizations between the first oligonucleotide and the target DNA, and between the second oligonucleotide and the target DNA, and is capable of improving the accuracy and the workability of a target nucleic acid detection method using the hybridization method.

Here, the term “at least a part of a region, of the antisense strand, which hybridizes with the second oligonucleotide is complementary to at least apart of a region, of the sense strand, which hybridizes with the first oligonucleotide” means that the region of the antisense strand which hybridizes with the second oligonucleotide and the region of the sense strand which hybridizes with the first oligonucleotide are able to partially form base pairs. The region of the sense strand which hybridizes with the first oligonucleotide and the region of the antisense strand which hybridizes with the second oligonucleotide do not always have to form complete base pairs, and respective region can be appropriately determined with a consideration of each hybridization efficiency.

That is, in a part of the target DNA, the sense strand of this part hybridizes with the first oligonucleotide, and the antisense strand of this part hybridizes with the second oligonucleotide. In the present invention, such a relation between the first oligonucleotide and the second oligonucleotide may be referred to as the “complementarity”.

In this manner, the first oligonucleotide and the second oligonucleotide have nucleotide sequences at least parts of which are complementary to each other. When these oligonucleotides are simultaneously added into one reaction solution, a hybrid between these oligonucleotides can be formed through hybridization. For this reason, when these are added into one reaction solution with the target DNA, the sense strand of the target DNA serving as the original target and the second oligonucleotide compete with each other for the first oligonucleotide in the hybridization. Similarly, the antisense strand of the target DNA serving as the original target and the first oligonucleotide compete with each other for the second oligonucleotide. However, those forming a hybrid between oligonucleotides are only a part of the oligonucleotides added to the reaction solution. Therefore, among the rest of the oligonucleotides which have not formed a hybrid between oligonucleotides, the first oligonucleotide can hybridize with the sense strand of the target DNA serving as the original target, and the second oligonucleotide can hybridize with the antisense strand of the target DNA serving as the original target, respectively. Utilizing this hybridization, the target DNA can be detected.

In addition, the region, of the first oligonucleotide, which hybridizes with the sense strand is not specifically limited as long as the region has a nucleotide sequence capable of hybridizing with the sense strand. It may be a region having a nucleotide sequence completely complementary to the sense strand, or a nucleotide sequence including mismatch(es). Similarly, the region, of the second oligonucleotide, which hybridizes with the antisense strand may be a region having a nucleotide sequence completely complementary to the antisense strand, or a nucleotide sequence including mismatch(es). Here, the term mismatch means a base which does not form a mutual base pair.

In particular, mismatches are preferably included in the respective oligonucleotides so that the number of mismatches in the hybrid between the first oligonucleotide and the second oligonucleotide is greater than the number of mismatches in the hybrid between the sense strand and the first oligonucleotide, or the number of mismatches in the hybrid between the antisense strand and the second oligonucleotide. The stability of the DNA hybridization is greatly dependent on the length of consecutive base pairs in the formed hybrid. It is considered that the thermodynamic stability of the hybrid is more impaired as the length of consecutive base pairs in the formed hybrid gets shorter. When mismatches are included, the formed hybrid is interrupted at the position(s) of the mismatches. Therefore, the hybrid becomes thermodynamically unstable, and its hybridization efficiency is lowered. Accordingly, by increasing the number of mismatches in the hybrid between oligonucleotides to more than the number of mismatches in the hybrid between the sense strand and the oligonucleotide, or the number of matches in the hybrid between the antisense strand and the oligonucleotide, it becomes possible to give pseudo-complementarity in which hybridization with the sense strand or the antisense strand can be performed preferentially to hybridization between oligonucleotides.

For example, in a case where the first oligonucleotide and the second oligonucleotide are each introduced with one mismatch so that their mismatches will not be overlapped in the hybrid therebetween, the hybrid between the oligonucleotide and the sense strand, or the hybrid between the oligonucleotide and the antisense strand would be mismatched at one point, while the hybrid between these oligonucleotides would be mismatched at two points. Therefore, the stability of the respective hybrids can be varied, so that pseudo-complementarity can be given to each oligonucleotide.

The number and the position(s) of mismatches to be introduced into each oligonucleotide are not specifically limited as long as the pseudo-complementarity can be retained. The number and the position(s) can be appropriately determined in consideration with the Tm values (melting temperatures of double-strands) and the hybridization efficiencies of these three types of formable hybrids (the hybrid between the first oligonucleotide and the second oligonucleotide, the hybrid between the sense strand and the first oligonucleotide, and the hybrid between the antisense strand and the second oligonucleotide), and the like. It is preferable to design and introduce one or more mismatch(es) into each of the first oligonucleotide and the second oligonucleotide so that their mismatches will not be overlapped in the hybrid. This is because the mismatch introduction into both oligonucleotides can more efficiently lower the thermodynamic stability of the hybrid between the oligonucleotides. However, if the numbers of introduced mismatches are too large, this may cause lowering in the hybridization efficiency with the sense strand or the antisense strand serving as the original target, and enhancement of hybridizations with other DNAs than the target DNA. Therefore, the number of mismatches is preferably 20% or less of the number of bases in a region of the oligonucleotide which hybridizes with the sense strand etc.

The bases constituting the first oligonucleotide and the second oligonucleotide of the present invention may be naturally-derived bases or synthesized bases including modified bases, although naturally-derived bases are preferred. This is because naturally-derived bases have less effect on the target DNA detection reactions such as hybridization, and can achieve easier and less expensive synthesis than synthesized bases.

The first oligonucleotide and the second oligonucleotide of the present invention may have an additional nucleotide sequence besides the region which hybridizes with the sense strand etc., as long as the hybridization with the sense strand etc. is not inhibited. Examples of such an additional sequence include restriction enzyme recognition sequences and sequences for labeling a nucleic acid.

Such oligonucleotides can be designed by any method well known in the art. For example, these can be easily designed by using publicly known genome sequence data or SNP data with a general primer design tool. Examples of such a primer design tool include Primer3 (Rozen, S., H. J. Skaletsky (1996), http://www-genome.wi.mit.edu/gonome_software/other/primer3.html) and Visual OMP (DNA Software Inc.) which are available on the web. Since such primer design tools can are capable of easy measurement of the Tm value and the hybridization efficiency, more preferable mismatches can be efficiently introduced. The publicly known genome sequence data is usually available on international nucleotide sequence databases, NCBI (National Center for Biotechnology Information), DDBJ (DNA Data Bank of Japan), and the like. In addition, the publicly known SNP data is available on databases such as a Japanese SNP database, JSNP (http://snp.ims.u-tokyo.ac.jp/index_ja.html) constructed by the Institute of Medical Science, the University of Tokyo.

The thus designed oligonucleotides can be synthesized by any method well known in the art. For example, they may be synthesized by a custom oligo synthesis service or may be synthesized by the user his/herself using a commercially available synthesizer.

In addition, the oligonucleotides for use in the target DNA detection method of the present invention may be labeled in order to facilitate the detection for the target DNA. The labeling substance is not specifically limited as long as it can be used for labeling nucleic acids. Examples thereof include radioisotopes, fluorescent substances, chemiluminescent substances, and biotin.

Using these hybrids obtained through respective hybridizations between the first oligonucleotide and the target DNA and between the second oligonucleotide and the target DNA, ligation reactions and/or PCR are performed, and then resultant products are detected, by which the target DNA can be detected. The detection method is not specifically limited as long as ligation reactions and/or PCR are employed. In addition, any publicly known technique can be employed as for the target nucleic acid detection method to which the hybridization method is applied. In particular, the technique is preferably for use in SNP detection.

Moreover, in this method, a plurality of oligonucleotides in a complementary relation may be simultaneously added into one reaction solution. Specifically, a third oligonucleotide and a fourth oligonucleotide mutually in a complementary relation may also be used in addition to the first oligonucleotide and the second oligonucleotide. Here, the third oligonucleotide refers to an oligonucleotide which hybridizes with the sense strand in a region different from the region of the sense strand which hybridizes with the first oligonucleotide, and the fourth oligonucleotide refers to an oligonucleotide which hybridizes with the antisense strand in a region different from the region of the antisense strand which hybridizes with the second oligonucleotide.

As to the method for performing PCR and detecting the resultant product, the target DNA can be detected by, for example, a method in which: using the first oligonucleotide as a forward primer and the third oligonucleotide as a reverse primer for amplification of the sense strand as a template, and using the fourth oligonucleotide as a forward primer and the second oligonucleotide as a reverse primer for amplification of the antisense strand as a template, PCR is performed in one reaction solution to simultaneously amplify the sense strand and the antisense strand of the target DNA, followed by detection of respective products.

Moreover, these products can be more accurately detected by checking the nucleotide sequence of each product.

As to the method for performing the ligation reactions and detecting the resultant products, the target DNA can be detected by, for example, a method in which: using the first oligonucleotide and the third oligonucleotide as probes which hybridize with adjacent regions of the sense strand, and using the second oligonucleotide and the fourth oligonucleotide as probes which hybridize with adjacent regions of the antisense strand, ligation reactions are performed in one reaction solution, followed by detection of the resultant products. Specifically, the sense strand and the antisense strand of the target DNA can be simultaneously detected by checking whether or not the oligonucleotide bound with the first oligonucleotide and the third oligonucleotide, and the oligonucleotide bound with the second oligonucleotide and the fourth oligonucleotide, can be respectively obtained.

As for the target DNA detection method of the present invention, it is particularly preferable to use the DigiTag2 assay (Non-patent Document 3) which excels in SNP detection.

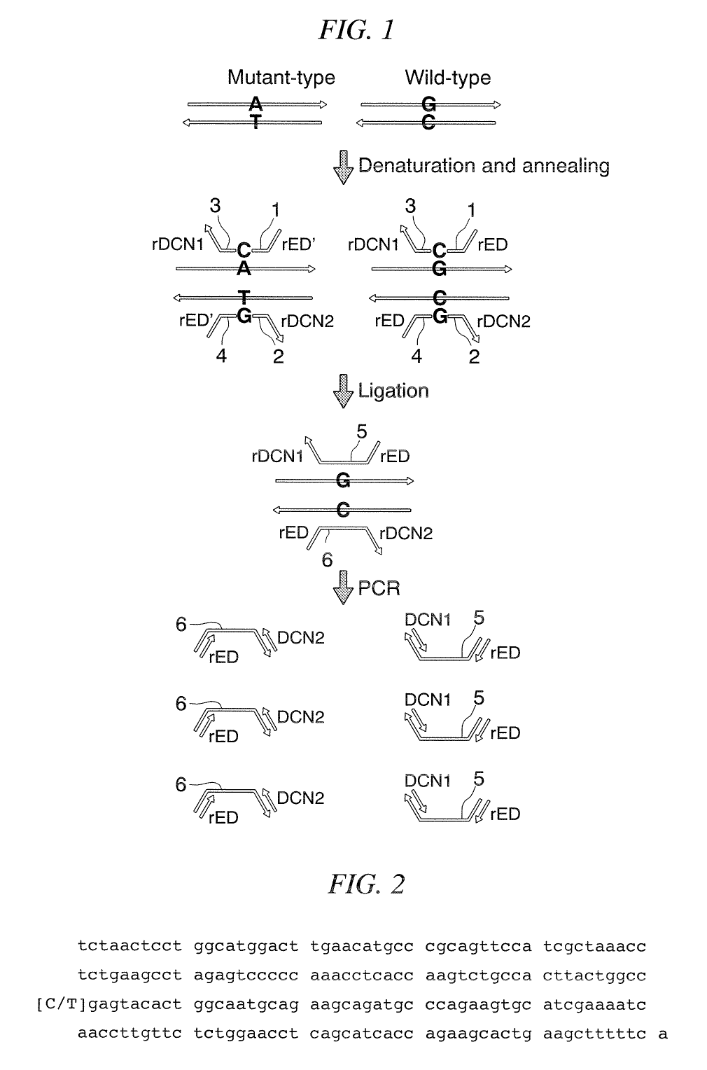

FIG. 1 shows a concept of one embodiment of the target DNA detection method of the present invention using the DigiTag2 assay. Specifically, it is a method for detecting whether the specimen DNA is the wild-type allele (target DNA) or not, assuming that the target nucleotide sequence is the nucleotide sequence of the wild-type allele of a SNP which is C in the wild-type allele and T in the mutant-type allele. For example, as the first oligonucleotide, a 5′ side (sense strand) probe 1 is used in which the base on the 3′ end is G that is complementary to the SNP of the sense strand of the wild-type allele (C), a region which hybridizes with the sense strand of the wild-type allele is located on the 3′ side, and rED serving as the tag sequence is located on the 5′ side. As the second oligonucleotide, a 3′ side (antisense strand) probe 2 is used in which the base on the 5′ end is a base that is complementary to the base adjacent to the 5′ side of the SNP of the antisense strand of the wild-type allele (G), a region which hybridizes with the antisense strand of the wild-type allele is located on the 5′ side, and rDCN2 serving as the tag sequence is located on the 3′ side. As the third oligonucleotide, a 3′ side (sense strand) probe 3 is used in which the base on the 5′ end is a base that is complementary to the base adjacent to the 5′ side of the SNP of the sense strand of the wild-type allele (C), a region which hybridizes with the sense strand of the wild-type allele is located on the 5′ side, and rDCN1 serving as the tag sequence is located on the 3′ side. As the fourth oligonucleotide, a 5′ side (antisense strand) probe 4 is used in which the base on the 3′ end is C that is complementary to the SNP of the antisense strand of the wild-type allele (G), a region which hybridizes with the antisense strand of the wild-type allele is located on the 3′ side, and rED serving as the tag sequence is located on the 5′ side.

These four types of oligonucleotides and the specimen DNA serving as the SNP detection target are added into one reaction solution, and subjected to denaturation process and annealing process. As a result, the sense strands of the wild-type allele and the mutant-type allele are hybridized with the 5′ side (sense strand) probe 1 and the 3′ side (sense strand) probe 3, while the antisense strands of the wild-type allele and the mutant-type allele are hybridized with the 3′ side (antisense strand) probe 2 and the 5′ side (antisense strand) probe 4. Here, hybrids are formed through respective hybridizations between the 5′ side (sense strand) probe 1 and the 3′ side (antisense strand) probe 2, and between the 3′ side (sense strand) probe 3 and the 5′ side (antisense strand) probe 4. However, since the pseudo-complementarity can be given through introduction of appropriate mismatches into respective oligonucleotides, respective oligonucleotides, even if added into one reaction solution, can be preferentially hybridized with the sense strand or the antisense strand.

Thereafter, a DNA ligase is added to the reaction solution to perform ligation reactions; by which, as shown in FIG. 1, if the specimen DNA is the wild-type, an oligonucleotide 5 bound with the 5′ side (sense strand) probe 1 and the 3′ side (sense strand) probe 3, and an oligonucleotide 6 bound with the 3′ side (antisense strand) probe 2 and the 5′ side (antisense strand) probe 4 can be obtained as the ligation products. When these ligation products are further subjected to PCR using a rED primer having a nucleotide sequence homologous to rED, a DCN1 primer having a nucleotide sequence complementary to rDCN1, and a DCN2 primer having a nucleotide sequence complementary to rDCN2, then amplified oligonucleotide 5 and oligonucleotide 6 can be obtained as the PCR products. On the other hand, if the specimen DNA is the mutant-type, oligonucleotides bound with these probes can neither be obtained as the ligation products, nor amplified by PCR. For this reason, the determination of the presence/absence of the PCR products enables the SNP detection of the specimen DNA. The amplification of the ligation products through real-time PCR enables rapid and semiquantitative SNP detection. In addition, the rED primer, the DCN1 primer, and the like for use in PCR may be labeled with a fluorescent substance etc., so as to facilitate the detection.

If an amplification of only either one of the oligonucleotide 5 and the oligonucleotide 6 is detected, a possibility of some problem in the series of operations, or a possibility of pseudo-positive can be considered. That is, the analysis of both of the sense strand and the antisense strand using oligonucleotides in a complementary relation can increase the accuracy of the target DNA detection.

In this manner, the target DNA detection method of the present invention is capable of rapid analysis of both sense strand and antisense strand of a target DNA through a single time detection reaction. In addition, the determination of the presence/absence of the detected target DNA through comparative examination of the obtained two analysis results can improve the accuracy and the success rate of the target DNA detection. For example, in a certain specimen, by determining that the specimen is positive only if both analysis results of the sense strand and the antisense strand are positive, that is, the analysis shows the target DNA has been detected, pseudo-positive cases can be excluded as much as possible, and therefore the accuracy of the target DNA detection can be improved. On the other hand, by determining that the specimen is positive in all cases determined to be positive by either of the analysis results of the sense strand or the antisense strand, all possibly positive specimens can be detected without a leak, and therefore the success rate of the target DNA detection can be improved. That is, in cases of the SNP detection, it is preferable for improving the accuracy, to utilize the base complementarity and to take the logical multiplication of two analysis results, and for improving the success rate, to focus on the signal distribution in a same polymorphism group and to take the logical addition of two analysis results.

Reagents such as nucleotides, buffer, and enzymes such as a DNA polymerase and a DNA ligase for use in the PCR and DigiTag2 assay can be those for use in usual PCR and DigiTag2 assay at usual concentrations. In addition, reaction conditions for the PCR and ligation reactions can be appropriately determined in consideration with the types of enzymes to be used, the Tm values of the oligonucleotides, and the like.

Moreover, a group of oligonucleotides for use in the target DNA detection method of the present invention can be set in one kit. For example, the first oligonucleotide and the second oligonucleotide can be set in one kit. The kit may also include the first oligonucleotide, the second oligonucleotide, the third oligonucleotide, and the fourth oligonucleotide. In addition, the kit preferably includes reagents such as nucleotides, buffer, and enzymes such as a DNA ligase and a DNA polymerase for ligation reactions and PCR. Furthermore, the kit may also include primers and probes for use in the detection of ligation products and PCR products. Use of such a kit enables rapid and easy detection for the target DNA.

Hereunder is a more detailed description of the present invention with reference to Examples. However, the present invention is not to be considered as being limited by these Examples.

### REFERENCE EXAMPLE 1

Probes usable for the SNP detection method with the DigiTag2 assay were designed with respect to a SNP in the human chromosome 16 whose NCBI accession number is rs17822931 (wild-type: C, mutant-type: T), to investigate the pseudo-complementary effect by the mismatch introduction. FIG. 2 shows the nucleotide sequence in the vicinity of rs17822931, wherein [C/T] denotes the SNP.

Under the setting that: the target nucleotide sequence was the nucleotide sequence of the sense strand of the rs17822931 wild-type allele; the first oligonucleotide was a common probe in which the base on the 3′ end was G that is complementary to the SNP(C), and a nucleotide sequence which hybridizes with the sense strand of the wild-type allele is held; and the second oligonucleotide was a query probe in which the base on the 5′ end was a base that is complementary to the base adjacent to the 5′ side of the SNP(G) of the antisense strand of the wild-type allele, and a nucleotide sequence which hybridizes with the antisense strand of the wild-type allele is held; respective probes were designed in a condition where the Tm value was 58° C. with use of Visual OMP Ver. 6. Furthermore, a common 2 probe in which a mismatch had been introduced in the 10th base from the 5′ end of the common probe, and a query 2 probe in which a mismatch had been introduced in the 4th base from the 3′ end of the query probe, were designed. The nucleotide sequences of the thus designed probes are shown in Table 1 respectively.

TABLE 1

| Probe | | |
|---|---|---|
| Common | GGCCAGTAAGTGGCAGACTTGGTGAGGTT | SEQ ID NO: 1 |
| Common 2 | GGCCAGTAACTGGCAGACTTGGTGAGGTT | SEQ ID NO: 2 |
| Query | GCCACTTACTGGCCC | SEQ ID NO: 3 |
| Query 2 | GCCACTTACTGCCCC | SEQ ID NO: 4 |

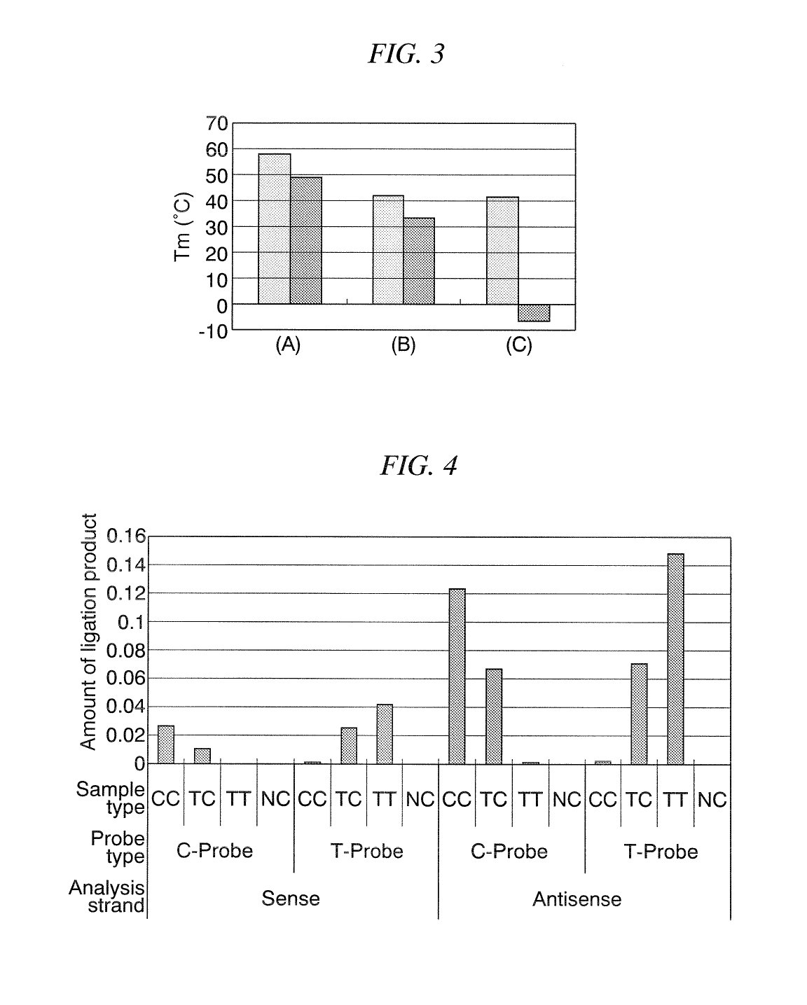

In order to obtain the respective Tm values for the hybrid between the common probe and the sense strand of the wild-type allele and the hybrid between the common probe and the query probe, variations due to the presence/absence of mismatch introduction were measured with use of Visual OMP Ver. 6. The results are shown in FIG. 3, wherein the left column shows the Tm value of the sense strand-common probe hybrid and the right column shows the Tm value of the common probe-query probe hybrid. In addition, (A) shows a case where the common probe and the query probe were used, (B) shows a case where the common probe and the query 2 probe were used, and (C) shows a case where the common 2 probe and the query 2 probe were used. That is, (A) is a case where the probes used had no mismatch, (B) is a case where only the query probe was introduced with a mismatch, and (C) is a case where both probes were introduced with mismatches.

As a result, the mismatch introduction into the query probe lowered the Tm values of both the sense strand-common probe hybrid and the common probe-query probe hybrid. In addition, further mismatch introduction into the common probe did not change the Tm value of the sense strand-common probe hybrid, but lowered the Tm value of the competitive common probe-query probe hybrid by about 40° C. That is, the results of FIG. 3 revealed that the mismatch introduction into both of the first oligonucleotide and the second oligonucleotide was able to improve the pseudo-complementarity between respective oligonucleotides, suggesting that simultaneous analysis of the sense strand and the antisense strand of the target DNA can be performed by the target DNA detection method of the present invention.

### EXAMPLES

With the target DNA detection method of the present invention using the DigiTag2 assay shown in FIG. 1, the SNP of rs17822931 was detected.

The wild-type homologous hybrid (C/C), the heterologous hybrid (C/T), and the mutant-type homologous hybrid (T/T) were used as DNA samples. Pure water without any DNA sample was used as the control.

Six types of probes shown in Table 2 were used as detection probes. In Table 2, three types on the top are probes for analyzing sense strands while three types on the bottom are probes for analyzing antisense strands. The “5′ side sense (C)” is a probe having the nucleotide sequence of SEQ ID NO: 5, in which the base on the 3′ end is G that is complementary to the wild-type SNP of the sense strand, a region which hybridizes with the sense strand of the wild-type allele is located on the 3′ side, and rED serving as the tag sequence is located on the 5′ side. The “5′ side sense (T)” is a probe having the nucleotide sequence of SEQ ID NO: 6, in which the base on the 3′ end is A that is complementary to the mutant-type SNP of the sense strand, a region which hybridizes with the sense strand of the mutant-type allele is located on the 3′ side, and rED serving as the tag sequence is located on the 5′ side. The “3′ side sense” is a probe having the nucleotide sequence of SEQ ID NO: 7, in which the base on the 5′ end is abase that is complementary to the base adjacent to the 5′ side of the SNP, a region which hybridizes with the sense strand is located on the 5′ side, and rDCN2 serving as the tag sequence is located on the 3′ side. In addition, the “5′ side antisense (C)” is a probe having the nucleotide sequence of SEQ ID NO: 8, in which the base on the 3′ end is C that is complementary to the wild-type SNP of the antisense strand, a region which hybridizes with the antisense strand of the wild-type allele is located on the 3′ side, and rED serving as the tag sequence is located on the 5′ side. The “5′ side antisense (T)” is a probe having the nucleotide sequence of SEQ ID NO: 9, in which the base on the 3′ end is T that is complementary to the mutant-type SNP of the antisense strand, a region which hybridizes with the antisense strand of the mutant-type allele is located on the 3′ side, and rED′ serving as the tag sequence is located on the 5′ side. The “3′ side antisense” is a probe having the nucleotide sequence of SEQ ID NO: 10, in which the base on the 5′ end is a base that is complementary to the base adjacent to the 5′ side of the SNP of the antisense strand, a region which hybridizes with the antisense strand is located on the 5′ side, and rDCN1 serving as the tag sequence is located on the 3′ side. Here, the 5′ side sense (C) probe, the 5′ side sense (T) probe, the 5′ side antisense (C) probe, and the 5′ side antisense (T) probe are probes in which a mismatch has been introduced in the 4th base from the 3′ end, similarly to the query 2 probe of Reference Example 1. In addition, the 3′ side sense probe is a probe in which a mismatch has been introduced in the 10th base from the 5′ end, and the 3′ side antisense probe is a probe in which mismatches have been respectively introduced in the 11th and 16th bases from the 5′ end. The nucleotide sequences of rED, rED′, rDCN1, and rDCN2 serving as the tag sequences are shown in Table 3.

TABLE 2

| Probe | |
|---|---|
| 5′ side | CCGTGTCCACTCTAGAAAAACCTGCATTGCCAGTGTAG |
| sense (C) | TCG |
| 5′ side | ACCACCGCTTGAATACAAAACATGCATTGCCAGTGTAG |
| sense (T) | TCA |
| 3′ side | GGCCAGTAACTGGCAGACTTGGTGAGGTTCATCTAAAG |
| sense | CGTTCCCAGTTCCA |
| 5′ side | CCGTGTCCACTCTAGAAAAACCTGCCACTTACTGCCCC |
| antisense | |
| (C) | |
| 5′ side | ACCACCGCTTGAATACAAAACATCTGCCACTTACTGCC |
| antisense | CT |
| (T) | |
| 3′ side | GAGTACACTGCCAATCCAGAAGCAGATGCCCGCAGATT |
| antisense | CATTGGTCAGAGAACA |

TABLE 3

| Name of | | |
|---|---|---|
| Tag sequence | | |
| rED | CCGTGTCCACTCTAGAAAA | SEQ ID NO: 11 |
| | ACCT | |
| rED′ | ACCACCGCTTGAATACAAA | SEQ ID NO: 12 |
| | ACAT | |
| rDCN1 | GCAGATTCATTGGTCAGAG | SEQ ID NO: 13 |
| | AACA | |
| rDCN2 | CATCTAAAGCGTTCCCAGT | SEQ ID NO: 14 |
| | TCCA | |

Theoretically, if the sense strand of the wild-type allele is present in the DNA sample, a ligation product resulting from the binding of the 5′ side sense (C) probe and the 3′ side sense probe is detected by the DigiTag2 assay. On the other hand, if the sense strand of the mutant-type allele is present in the DNA sample, a ligation product resulting from the binding of the 5′ side sense (T) probe and the 3′ side sense probe is detected. Similarly, if the antisense strand of the wild-type allele is present, a ligation product resulting from the binding of the 5′ side antisense (C) probe and the 3′ side antisense probe is detected, while, if the antisense strand of the mutant-type allele is present, a ligation product resulting from the binding of the 5′ side antisense (T) probe and the 3′ side antisense probe is detected.

First, DNA fragments containing SNP sites were amplified by PCR using a forward primer having the nucleotide sequence of SEQ ID NO: 15 and a reverse primer reverse having the nucleotide sequence of SEQ ID NO: 16. Specifically, 10 μL of a PCR solution was prepared by adding 5 ng of a DNA sample, and the forward and the reverse primers at each final concentration of 0.1 μM into 5 μL of 2×QIAGEN Multiplex PCR Master Mix (QIAGEN). PCR was performed by treating the PCR solution at 95° C. for 15 seconds, followed by forty thermal cycles of 95° C. for 30 seconds and 68° C. for 6 minutes.

Next, to a 100-fold diluted solution of the obtained PCR product was simultaneously added the six types of probes shown in Table 2. The mixture was subjected to denaturation and annealing, followed by ligation reactions. Specifically, to 1 μL of the PCR product dilution solution was added 8 U Taq ligase, respective probes at each final concentration of 333 pM, and the balance of pure water until the reaction solution reached a final volume of 15 μL. The reaction solution was subjected to a denaturation treatment at 95° C. for 1 minute and a subsequent treatment at 58° C. for 15 minutes to effect hybridizations between the respective probes and the DNA sample, followed by ligation reactions.

Then, a real-time PCR was further performed using the rED primer having a nucleotide sequence homologous to the tag sequence rED and the DCN2 primer having a nucleotide sequence complementary to the tag sequence rDCN2, by which a ligation product resulting from the binding of the 5′ side sense (C) probe and the 3′ side sense probe was detected. Similarly, a real-time PCR was performed using the rED′ primer having a nucleotide sequence homologous to the tag sequence rED′ and the DCN2 primer, by which a ligation product resulting from the binding of the 5′ side sense (T) probe and the 3′ side sense probe was detected. Furthermore, a real-time PCR was performed using the rED primer and the DCN1 primer having a nucleotide sequence complementary to the tag sequence rDCN1, by which a ligation product resulting from the binding of the 5′ side antisense (C) probe and the 3′ side antisense probe was detected. In addition, a real-time PCR was performed using the rED′ primer and the DCN1 primer, by which a ligation product resulting from the binding of the 5′ side antisense (T) probe and the 3′ side antisense probe was detected.

Specifically, 20 μL of a real-time PCR solution was prepared by adding 1 μL of a ligation product, and respective primers at each final concentration of 0.1 μM into 10 μL of 2×SYBR Premix (TaKaRa). Real-time PCR was performed by treating the real-time PCR solution at 95° C. for 10 seconds, followed by sixty thermal cycles of 95° C. for 5 seconds and 61° C. for 7 seconds. The amounts of respective ligation products were relatively measured.

FIG. 4 shows the thus obtained measurement results, wherein the “Sample type” means the type of SNP of the DNA sample used, and the “NC” means the control. In addition, the “Probe type” means the SNP type serving as the analysis target for the probe used, the “C-Probe” means that either one of the 5′ side sense (C) probe or the 5′ side antisense (C) probe was used, and the “T-Probe” means that either one of the 5′ side sense (T) probe or the 5′ side antisense (T) probe was used. With the 5′ side sense (C) probe for detecting the sense strand of the wild-type SNP, the detection amount of the ligation product was the largest when the SNP type of the DNA sample was the wild-type homologous hybrid, followed by the heterologous hybrid which showed the second largest detection amount, whereas no ligation product was detected in the mutant-type homologous hybrid and the control. With the 5′ side sense (T) probe for detecting the sense strand of the mutant-type SNP, the detection amount of the ligation product was the largest when the SNP type of the DNA sample was the mutant-type homologous hybrid, followed by the heterologous hybrid which showed the second largest detection amount, whereas no ligation product was detected in the wild-type homologous hybrid and the control. A similar tendency was observed in the analyses of the antisense strand. That is, in all measurement results, the amounts of the ligation products were remarkably increased in cases where the SNP type of the DNA sample was the same as the type of the detection probe, as compared to cases where the SNP type of the DNA sample was different from the type of the detection probe, showing that the SNP determination results on the basis of the amount of the ligation product agreed with the actual SNP type. Accordingly, these results revealed that simultaneous analysis of the sense strand and the antisense strand of the target DNA can be performed by the target DNA detection method of the present invention.

### INDUSTRIAL APPLICABILITY

The target DNA detection method of the present invention enables simultaneous analysis of the sense strand and the antisense strand of a target DNA by applying the hybridization method so that the target DNA can be easily and highly accurately detected. Therefore, this method can be utilized particularly in the field of gene analysis such as SNP detection, and the like.

1. A method for detecting a target DNA composed of a sense strand having a target nucleotide sequence and an antisense strand complementary to the sense strand, comprising the steps of:
providing a first oligonucleotide which hybridizes with the sense strand, and a second oligonucleotide which hybridizes with the antisense strand; wherein
at least a part of a region, of the antisense strand, which hybridizes with the second oligonucleotide is complementary to at least a part of a region, of the sense strand, which hybridizes with the first oligonucleotide; and
adding the first oligonucleotide and the second oligonucleotide are simultaneously into one reaction solution to effect respective hybridizations with the target DNA; and subjecting the reaction solution to ligation reactions and/or PCR (Polymerase Chain Reaction), thereby detecting resultant products of the ligation and/or PCR.

2. A target DNA detection method according to claim 1, wherein the number of mismatches in the hybrid between said first oligonucleotide and said second oligonucleotide is greater than the number of mismatches in the hybrid between said sense strand and said first oligonucleotide, or the number of mismatches in the hybrid between said antisense strand and said second oligonucleotide.

3. A target DNA detection method according to claim 2, wherein one or more mismatches are respectively introduced into said first oligonucleotide and said second oligonucleotide.

4. A target DNA detection method according to claim 1, wherein said target nucleotide sequence is a nucleotide sequence containing a SNP (Single Nucleotide Polymorphism) site.

5. A method for detecting a target DNA composed of a sense strand having a target nucleotide sequence and an antisense strand complementary to the sense strand, comprising the steps of:
providing
a first oligonucleotide which hybridizes with the sense strand, and a second oligonucleotide which hybridizes with the antisense strand, wherein at least a part of a region, of the antisense strand, which hybridizes with the second oligonucleotide is complementary to at least a part of a region, of the sense strand, which hybridizes with the first oligonucleotide; and
a third oligonucleotide which hybridizes with said sense strand in a region different from said region of the sense strand which hybridizes with the first oligonucleotide, and a fourth oligonucleotide which hybridizes with said antisense strand in a region different from said region of the antisense strand which hybridizes with the second oligonucleotide, wherein
at least a part of a region, of the antisense strand, which hybridizes with the fourth oligonucleotide is complementary to at least a part of a region, of the sense strand, which hybridizes with the third oligonucleotide; and
adding the first oligonucleotide, the second oligonucleotide, the third oligonucleotide, and the fourth oligonucleotide simultaneously into one reaction solution to effect respective hybridizations with the target DNA; subjecting the reaction solution to ligation reactions and/or PCR (Polymerase Chain Reaction), thereby detecting resultant products of the ligation and/or PCR.

6. A target DNA detection kit comprising a first oligonucleotide which hybridizes with the sense strand of the target DNA, and a second oligonucleotide which hybridizes with the antisense strand of the target DNA, wherein at least parts of said first and said second oligonucleotide are complementary to each other.

7. A target DNA detection kit comprising a first oligonucleotide which hybridizes with the sense strand of the target DNA, a second oligonucleotide which hybridizes with the antisense strand of the target DNA, a third oligonucleotide which hybridizes with said sense strand of the target DNA in a region different from said region of the sense strand which hybridizes with the first oligonucleotide, and a fourth oligonucleotide which hybridizes with said antisense strand of the target DNA in a region different from said region of the antisense strand which hybridizes with the second oligonucleotide, wherein at least parts of said first and said second oligonucleotides are complementary to each other, and at least parts of said third and said fourth oligonucleotides are complementary to each other.
